(19) United States
(12) Patent Application Publication
(10) Pub. No.: US 2022/0227772 A1
(43) Pub. Date: Jul. 21, 2022

(54) DEUTERATED TRIAZOLOPYRIMIDINES

(71) Applicants: THE TRUSTEES OF THE UNIVERSITY OF PENNSYLVANIA, Philadelphia, PA (US); THE REGENTS OF THE UNIVERSITY OF CALIFORNIA, Oakland, CA (US)

(72) Inventors: Carlo BALLATORE, San Diego, CA (US); Kurt R. BRUNDEN, Media, PA (US); Killian OUKOLOFF, San Diego, CA (US); Virgina M.Y. LEE, Philadelphia, PA (US); John Q. TROJANOWSKI, Philadelphia, PA (US); Amos B. SMITH, III, Merion, PA (US)

(21) Appl. No.: 17/609,089

(22) PCT Filed: May 7, 2020
(86) PCT No.: PCT/US20/31814

§ 371 Date: Nov. 5, 2021

Related U.S. Application Data
(60) Provisional application No. 62/844,276, filed on May 7, 2019.

(51) Int. Cl. C07D 487/04; A61P 25/28

(52) U.S. Cl. CPC C07D 487/04; A61P 25/28

(57) ABSTRACT

The present disclosure provides compounds of formula (I) or a pharmaceutically acceptable salt or stereoisomer thereof, wherein R¹-R⁶ are defined herein. Also provided are compositions comprising a compound described herein and a pharmaceutically effective excipient, methods of stabilizing microtubules in a patient comprising administering to the patient a microtubule-stabilizing amount of a compound described herein, methods of treating cancer in a patient comprising administering to the patient a therapeutically effective amount of a compound described herein, and methods of treating a neurodegenerative disease in a patient comprising administering to the patient a therapeutically effective amount of a compound described herein.

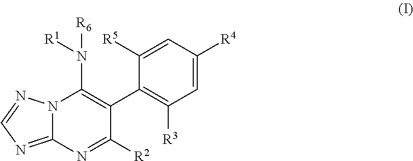

### CROSS-REFERENCE TO RELATED APPLICATIONS

This application claims priority to U.S. Provisional Patent Application No. 62/844,276, filed May 7, 2019, the disclosure of which is incorporated by reference herein in its entirety.

### STATEMENT REGARDING FEDERALLY SPONSORED RESEARCH OR DEVELOPMENT

This invention was made with Government support under grant AG044332 awarded by the National Institutes of Health. The Government has certain rights in the invention.

### TECHNICAL FIELD

The disclosure relates to compounds and methods for treating neurodegenerative diseases.

### BACKGROUND

Agents that affect microtubule (MT) structure have proven useful for the treatment of cancers, as exemplified by drugs such as paclitaxel, vinblastine and vincristine. Such drugs affect cancer cell division by interfering with normal MT structure and function during cell division, where MTs play a critical role in chromosomal segregation during mitosis. MT-active [1,2,4]triazolo[1,5-a]pyrimidines and related heterocyclic molecules have attracted attention as potential candidates for a variety of applications including cancer chemotherapy, as well as neurodegenerative disease treatment. Neurodegenerative diseases such as Alzheimer's disease and related tauopathies are characterized by disengaged tau proteins from MTs resulting in destabilized MTs in brain.

Additional microtubule-active compounds which affect MT structure are needed.

### SUMMARY

The present disclosure provides compounds of formula (I), wherein R¹-R⁶ are defined herein:

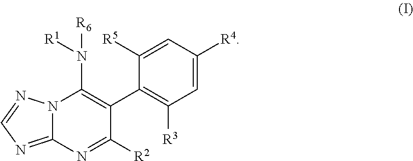

The present disclosure also provides compounds of formula (II), (III), (IV), or (V), wherein R¹-R⁵ and R⁷ are defined herein:

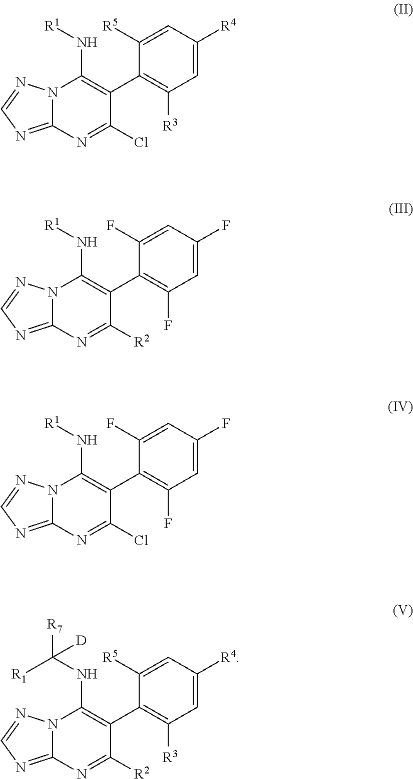

The present disclosure further provides compositions comprising a compound as described herein and a pharmaceutically acceptable excipient.

The present disclosure also provides methods of stabilizing microtubules in a patient comprising administering to the patient a microtubule-stabilizing amount of a compound as described herein.

The present disclosure additionally provides methods of treating a neurodegenerative disease in a patient comprising administering to the patient a therapeutically effective amount of a compound described herein. In some embodiments, the neurodegenerative disease is characterized by a tauopathy or compromised microtubule function in the brain of the patient.

The present disclosure further provides methods of treating a cancer in a patient comprising administering to the patient a therapeutically effective amount of a compound described herein.

Other aspects and embodiments of the invention will be readily apparent from the following detailed description of the invention.

### DETAILED DESCRIPTION OF THE PREFERRED EMBODIMENTS

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

The term “alkyl,” when used alone or as part of a substituent group, refers to a straight- or branched-chain alkyl group having from 1 to 12 carbon atoms (“C₁₋₁₂”), preferably 1 to 6 carbons atoms (“C₁₋₆”), in the chain. Examples of alkyl groups include methyl (Me, C₁alkyl) ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and groups that in light of the ordinary skill in the art and the teachings provided herein would be considered equivalent to any one of the foregoing examples. An alkyl moiety is optionally substituted with one, two, or three substituents selected from halo (F, Cl, Br, or I, preferably F), —OH, —OC₁₋₆alkyl, —CN, —NH₂, —NH(C₁₋₆alkyl), —NH(C₁₋₆alkyl)₂, C₃₋₈cycloalkyl, heterocyclyl, aryl, or heteroaryl.

The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “-C₀alk-” refers to a bond.

The terms “haloalkyl” and “halogenated alkyl” are interchangeable and, when used alone or as part of a substituent group, refer to an alkyl group as described above having one, two, or three halogen atoms attached to a single carbon atom. Preferably, the halogen is F. In some embodiments, haloalkyl includes perfluoroalkyl groups whereby the alkyl group is terminated with a CF₃, CH₂F, or CHF₂. Examples of haloalkyl groups include CF₃, CHF₂, CH₂F, CH₂CF₃, CHFCF₃, CF₂CF₃, CH₂CHF₂, CH₂CH₂F, CHFCH₃, CF₂CH₃, CHFCHF₂, CF₂CHF₂, among others, and groups that in light of the ordinary skill in the art and the teachings provided herein would be considered equivalent to any one of the foregoing examples. A haloalkyl moiety is optionally substituted with one, two, or three substituents selected from —OH, —OC₁₋₆alkyl, —CN, —NH₂, —NH(C₁₋₆alkyl), —NH(C₁₋₆alkyl)₂, C₃₋₈cycloalkyl, heterocyclyl, aryl, or heteroaryl.

The term “alkoxy,” when used alone or as part of a substituent group, refers to a straight- or branched-chain alkoxy group, i.e., O-alkyl, having from 1 to 12 carbon atoms (“C₁₋₁₂”), preferably 1 to 6 carbons atoms (“C₁₋₆”), in the chain. Examples of alkoxy groups include methoxy (OMe, C₁alkoxy) ethoxy (OEt, C₂alkoxy), n-propoxy (OⁿPr, C₃alkoxy), isopropoxy (OⁱPr, C₃alkoxy), butoxy (OBu, C₄alkoxy), isobutoxy (OⁱBu, C₄alkoxy), sec-butoxy (O^{s}Bu, C₄alkoxy), tert-butoxy (O^{t}Bu, C₄alkoxy), pentoxy (C₅alkoxy), isopentoxy (C₅alkoxy), tert-pentoxy (C₅alkoxy), hexoxy (C₆alkoxy), isohexoxy (C₆alkoxy), and groups that in light of the ordinary skill in the art and the teachings provided herein would be considered equivalent to any one of the foregoing examples. An alkoxy moiety is optionally substituted with one, two, or three substituents selected from halo (F, Cl, Br, or I, preferably F), —OH, —CN, —NH₂, —NH(C₁₋₆alkyl), —NH(C₁₋₆alkyl)₂, C₃₋₈cycloalkyl, heterocyclyl, aryl, or heteroaryl.

“Heterocyclyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18: refers to each integer in the given range, e.g., “3 to 18 ring atoms” means that the heterocyclyl group may consist of 3 ring atoms, 4 ring atoms, etc., up to and including 18 ring atoms. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of heterocyclyl radicals include, but are not limited to, azepanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. “Heterocyclyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic. A heterocyclyl moiety is optionally substituted with one, two, or three substituents selected from halo (F, Cl, Br, or I, preferably F), —OH, —CN, —NH₂, —NH(C₁₋₆alkyl), —NH(C₁₋₆alkyl)₂, C₃₋₈cycloalkyl, heterocyclyl, aryl, or heteroaryl.

The term “cycloalkyl” refers to monocyclic, non-aromatic hydrocarbon groups having from 3 to 10 carbon atoms (“C₃₋₁₀”), preferably from 3 to 6 carbon atoms (“C₃₋₆”). Examples of cycloalkyl groups include, for example, cyclopropyl (C₃), cyclobutyl (C₄), cyclopentyl (C₅), cyclohexyl (C₆), 1-methylcyclopropyl (C₄), 2-methylcyclopentyl (C₄), adamantanyl (C₁₀), and the like. A cycloalkyl is optionally substituted with one, two, or three substituents selected from halo (F, Cl, Br, or I, preferably F), —OH, —CN, —NH₂, —NH(C₁₋₆alkyl), —NH(C₁₋₆alkyl)₂, C₃₋₈cycloalkyl, heterocyclyl, aryl, or heteroaryl.

The term “aryl” refers to carbocyclic aromatic groups having from 6 to 10 carbon atoms (“C₆₋₁₀”) such as phenyl, naphthyl, and the like. An aryl is optionally substituted with one, two, or three substituents selected from halo (F, Cl, Br, or I, preferably F), —OH, —OC₁₋₆alkyl, —CN, —NH₂, —NH(C₁₋₆alkyl), —NH(C₁₋₆alkyl)₂, C₃₋₈cycloalkyl, heterocyclyl, aryl, or heteroaryl. In some embodiments, the aryl is substituted with one halo. In other embodiments, the aryl is substituted with one F. In still other embodiments, the aryl is phenyl and is optionally substituted with one halo. In yet further embodiments, the aryl is phenyl and is optionally substituted with one F.

“Heteroaryl” refers to a 5- to 18-membered aromatic radical, e.g., C₅₋₁₈heteroaryl, that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range, e.g., “5 to 18 ring atoms” means that the heteroaryl group may contain 5 ring atoms, 6 ring atoms, etc., up to and including 18 ring atoms. An N-containing heteroaryl moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). A heteroaryl is optionally substituted with one, two, or three substituents selected from halo (F, Cl, Br, or I, preferably F), —OH, —CN, —NH₂, —NH(C₁₋₆alkyl), −NH(C₁₋₆alkyl)₂, C₃₋₈cycloalkyl, heterocyclyl, aryl, or heteroaryl.

When a range of carbon atoms is used herein, for example, C₁₋₆, all ranges, as well as individual numbers of carbon atoms are encompassed. For example, “C₁₋₃” includes C₁₋₃, C₁₋₂, C₂₋₃, C₁, C₂, and C₃.

The terms “halogen” and “halo” represent chlorine, fluorine, bromine, or iodine. The term “halo” represents chloro, fluoro, bromo, or iodo.

“Compounds of the present disclosure,” and equivalent expressions, are meant to embrace compounds of the Formulae (I), (II), (III), (IV), or (V) as described herein, which expression includes the pharmaceutically acceptable salts, where the context so permits. Similarly, reference to intermediates, whether or not they themselves are claimed, is meant to embrace their salts, and solvates, where the context so permits. Similarly, the term “compound(s) of formula (I)” includes those compounds of “formula (I),” as well as compounds of any of the formula (I) subgenera.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

The terms “patient” or “subject” as used herein refer to a mammalian animal and are used interchangeably. In some embodiments, the patient or subject is a human. In other embodiments, the patient or subject is a veterinary or farm animal, a domestic animal or pet, or animal normally used for clinical research.

“Treating” any disease or disorder refers, in some embodiments, to ameliorating the disease or disorder (i.e., arresting or reducing the development of the disease or at least one of the clinical symptoms thereof). In some embodiments, “treating” or “treatment” refers to ameliorating at least one physical parameter, which may not be discernible by the subject. In other embodiments, “treating” or “treatment” refers to modulating the disease or disorder, either physically, (e.g., stabilization of a discernible symptom), physiologically, (e.g., stabilization of a physical parameter), or both. In further embodiments, “treating” or “treatment” refers to delaying the onset of the disease or disorder.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)-or (S)-stereoisomers or as mixtures thereof.

Unless indicated otherwise, the description or naming of a particular compound in the specification and claims is intended to include both individual enantiomers and mixtures, racemic or otherwise, thereof. Within the present disclosure, any open valency appearing on a carbon, oxygen, or nitrogen atom in any structure described herein indicates the presence of a hydrogen atom. Where a chiral center exists in a structure, but no specific stereochemistry is shown for that center, both enantiomers, separately or as a mixture, are encompassed by that structure. The methods for the determination of stereochemistry and the separation of stereoisomers are well-known in the art.

The [1,2,4]triazolo[1,5-a]pyrimidine compounds described herein either promote stabilization of MTs or disrupt MT integrity. In some embodiments, the compounds are of formula (I) or a pharmaceutically acceptable salt or stereoisomer thereof. In further embodiments, the disclosure provides a pharmaceutically acceptable salt of a compound of formula (I). In other embodiments, the disclosure provides a stereoisomer of a compound of formula (I).

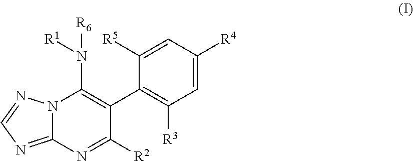

In these compounds, R¹ is C₁₋₁₂alkyl, C₃₋₈cycloalkyl, C₁₋₆alk-C₃₋₈cycloalkyl, or C₁₋₆alk-C₃₋₈cycloheteroalkyl and is substituted by one or more deuterium. In some embodiments, R¹ is substituted by one, two, or three deuteriums. In other embodiments, R¹ is substituted by one deuterium. In further embodiments, R¹ is substituted by two deuteriums. In still other embodiments, R¹ is substituted by three deuteriums.

In some aspects, R¹ is C₁₋₁₂alkyl substituted by one or more deuterium. In some embodiments, R¹ is deuterated methyl. In other embodiments, R¹ is deuterated ethyl. In further embodiments, R¹ is deuterated propyl. In yet other embodiments, R¹ is deuterated butyl. In still further embodiments, R¹ is deuterated pentyl. In other embodiments, R¹ is deuterated hexyl. In further embodiments, R¹ is deuterated heptyl. In still other embodiments, R¹ is deuterated octyl. In yet further embodiments, R¹ is deuterated nonyl. In other embodiments, R¹ is deuterated decyl. Preferably R¹ is deuterated pentyl or deuterated hexyl. In further embodiments, R¹ is:

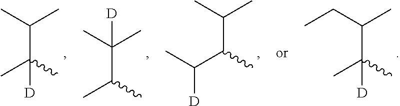

In other aspects, R¹ is C₃₋₈cycloalkyl substituted by one or more deuterium. In some embodiments, R¹ is deuterated cyclopropyl. In other embodiments R¹ is deuterated cyclobutyl. In further embodiments, R¹ is deuterated cyclopentyl. In still other embodiments, R¹ is deuterated cyclohexyl. In yet further embodiments, R¹ is deuterated cycloheptyl. In other embodiments, R¹ is deuterated cyclooctyl. In further embodiments, R¹ is deuterated bicyclo[1.1.1]pentyl. Preferably, R¹ is deuterated cyclopropyl or deuterated bicyclo[1.1.1]pentyl. In other embodiments, R¹ is:

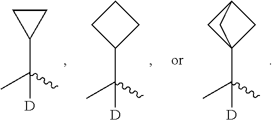

In further aspects, R¹ is C₁₋₆alk-C₃₋₈cycloalkyl substituted by one or more deuterium. In some embodiments, R¹ is deuterated —CH₂-cyclopropyl. In other embodiments, R¹ is deuterated —CH₂-cyclobutyl. In further embodiments, R¹ is deuterated —CH₂-cyclopentyl. In still other embodiments, R¹ is deuterated —CH₂-cyclohexyl. In yet further embodiments, R¹ is deuterated —CH₂-cycloheptyl. In other embodiments, R¹ is deuterated —CH₂-cyclooctyl. In further embodiments, R¹ is deuterated —CH₂-bicyclo[1.1.1]pentyl. Preferably R¹ is deuterated —CH₂-cyclopropyl or deuterated —CH₂-bicyclo[1.1.1]pentyl.

In yet other aspects, R¹ is C₁₋₆alk-C₃₋₈cycloheteroalkyl substituted by one or more deuterium. In some embodiments, R¹ is deuterated —CH₂-aziridinyl. In other embodiments, R¹ is deuterated —CH₂-oxiranyl. In further embodiments, R¹ is deuterated —CH₂-thiiranyl. In still other embodiments, R¹ is deuterated —CH₂-azetidinyl. In yet further embodiments, R¹ is deuterated —CH₂-oxetanyl. In other embodiments, R¹ is deuterated —CH₂-thietanyl. In further embodiments, R¹ is deuterated —CH₂-pyrrolidinyl. In yet other embodiments, R¹ is deuterated —CH₂-tetrahydrothiophenyl. In still further embodiments, R¹ is deuterated —CH₂-tetrahydrofuryl.

R¹ as defined may also be further substituted by one or more F atoms. In some embodiments, R¹ is further substituted by 1 to 3 F atoms. In further embodiments, R¹ is further substituted by 1 F atom. In other embodiments, R¹ is:

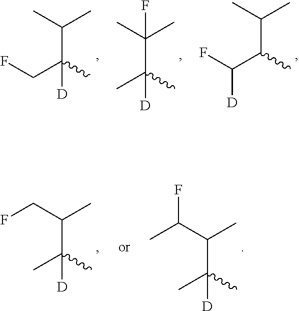

In further embodiments, R¹ is:

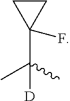

In yet further embodiments, R¹ is further substituted by 2 F atoms. In still other embodiments, R¹ is:

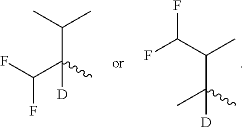

In other embodiments, R¹ is further substituted by 3 F atoms. In further embodiments, R¹ is:

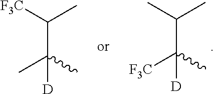

In yet other embodiments, R¹ is:

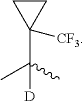

R² is H, Br, Cl, F, CH₃, or CF₃. In some embodiments, R² is H. In other embodiments, R² is Br, Cl, or F. In further embodiments, R² is Cl. In yet other embodiments, R² is Br. In still further embodiments, R² is F. In yet embodiments, R² is CH₃. In further embodiments, R² is CF₃.

R³ is H, Br, Cl, or F. In some embodiments, R³ is H. In other embodiments, R³ is Br, Cl, or F. In further embodiments, R³ is Br. In yet other embodiments, R³ is Cl. In still further embodiments, R³ is F.

R⁴ is H, Br, Cl, F, CN, CF₃, or CF₂C₁₋₆alkyl. In some embodiments, R⁴ is H. In other embodiments, R⁴ is Br, Cl, or F. In further embodiments, R⁴ is Br. In yet other embodiments, R⁴ is Cl. In still further embodiments, R⁴ is F. In other embodiments, R⁴ is CN. In further embodiments, R⁴ is CF₃. In still other embodiments, R⁴ is CF₂C₁₋₆alkyl. In yet further embodiments, R⁴ is CF₂C₁alkyl, CF₂C₂alkyl, CF₂C₃alkyl, CF₂C₄alkyl, CF₂C₅alkyl, or CF₂C₆alkyl.

R⁵ is H, Br, Cl, or F. In some embodiments, R⁵ is H. In other embodiments, R⁵ is Br, Cl, or F. In further embodiments, R⁵ is Br. In yet other embodiments, R⁵ is Cl. In still other embodiments, R⁵ is F.

R⁶ is H, C₁₋₁₂alkyl, C₃₋₈cycloalkyl, C₁₋₆alk-C₃₋₈cycloalkyl, C₁₋₆alk-C₃₋₈cycloheteroalkyl, diazirinyl, halogenated C₁₋₁₂alkyl, C₁₋₁₂alkyl substituted with diazirinyl, aryl substituted with diazirinyl, or C(O)(aryl substituted with diazirinyl). In some embodiments, R⁶ is H. In other embodiments, R⁶ is C₁₋₁₂alkyl. In some aspects, R⁶ is methyl. In other aspects, R⁶ is ethyl. In further aspects, R⁶ is propyl. In yet other aspects, R⁶ is butyl. In still further aspects, R⁶ is pentyl. In other aspects, R⁶ is hexyl. In further aspects, R⁶ is heptyl. In still other aspects, R⁶ is octyl. In yet further aspects, R⁶ is nonyl. In other aspects, R⁶ is decyl. In further embodiments, R⁶ is C₃₋₈cycloalkyl. In still other embodiments, R⁶ is C₁₋₆alk-C₃₋₈cycloalkyl. In yet further embodiments, R⁶ is C₁₋₆alk-C₃₋₈cycloheteroalkyl. In still other embodiments R⁶ is halogenated C₁₋₁₂alkyl. In further embodiments, R⁶ is C₁₋₁₂alkyl substituted with diazirinyl. In still other embodiments, R⁶ is aryl substituted with diazirinyl. In yet further embodiments, R⁶ is C(O)(aryl substituted with diazirinyl).

In some embodiments, the disclosure provides compounds of formula (II), wherein R¹ and R³-R⁵ are defined herein.

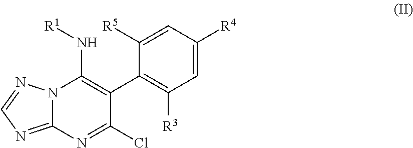

In other embodiments, the disclosure provides compounds of formula (III), wherein R¹ and R² are defined herein.

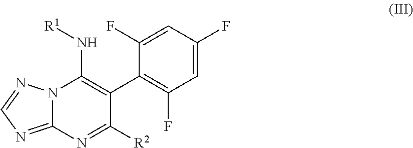

In further embodiments, the disclosure provides compounds of formula (IV), wherein R¹ is defined herein.

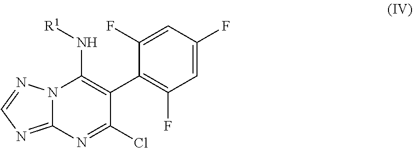

In still other embodiments, the disclosure provides compounds of formula (V).

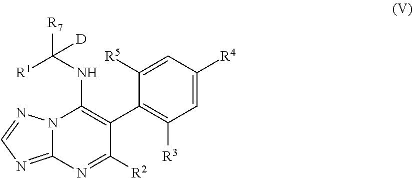

In this structure, R¹-R⁵ are defined herein and R⁷ is H, C₁₋₆alkyl, C₃₋₈cycloalkyl, C₁₋₆alk-C₃₋₈cycloalkyl, or C₁₋₆alk-C₃₋₈cycloheteroalkyl. In some embodiments, R⁷ is H. In other embodiments, R⁷ is C₁₋₆alkyl. In some aspects, R⁷ is methyl. In other aspects, R⁷ is ethyl. In further aspects, R⁷ is propyl. In yet other aspects, R⁷ is butyl. In still further aspects, R⁷ is pentyl. In other aspects, R⁷ is hexyl. In further embodiments, R⁷ is C₃₋₈cycloalkyl. In some aspects, R⁷ is cyclopropyl. In other aspects, R⁷ is cyclobutyl. In further aspects, R⁷ is cyclopentyl. In yet other aspects, R⁷ is cyclohexyl. In still further aspects, R⁷ is cycloheptyl. In other aspects, R⁷ is cyclooctyl. In yet other embodiments, R⁷ is C₁₋₆alk-C₃₋₈cycloalkyl. In some aspects, R⁷ is —CH₂-cyclopropyl. In other aspects, R⁷ is —CH₂-cyclobutyl. In further aspects, R⁷ is —CH₂-cyclopentyl. In still other aspects, R⁷ is —CH₂-cyclohexyl. In yet further aspects, R⁷ is —CH₂-cycloheptyl. In other aspects, R⁷ is —CH₂-cyclooctyl. In further aspects, R⁷ is deuterated —CH₂-bicyclo[1.1.1]pentyl. In still further embodiments, R⁷ is C₁₋₆alk-C₃₋₈cycloheteroalkyl. In some aspects, R⁷ is —CH₂-aziridinyl. In other aspects, R¹ is —CH₂-oxiranyl. In further aspects, R⁷ is —CH₂-thiiranyl. In still other aspects, R⁷ is —CH₂-azetidinyl. In yet further aspects, R⁷ is —CH₂-oxetanyl. In other aspects, R⁷ is —CH₂-thietanyl. In further aspects, R⁷ is —CH₂-pyrrolidinyl. In yet other aspects, R⁷ is —CH₂-tetrahydrothiophenyl. In still further embodiments, R⁷ is —CH₂-tetrahydrofuryl.

In yet other embodiments, the compound is:

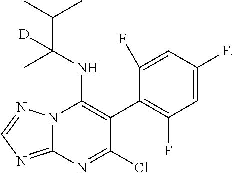 [SMILES: [2H]C(C)(Nc1c(-c2c(F)cc(F)cc2F)c(Cl)nc2ncnn12)C(C)C]

In further embodiments, the compound is:

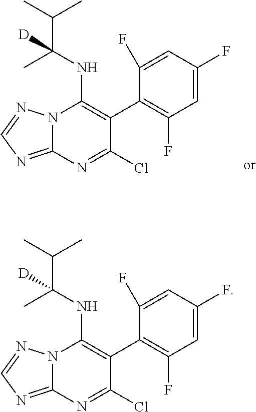 [SMILES: [2H][C@](C)(Nc1c(-c2c(F)cc(F)cc2F)c(Cl)nc2ncnn12)C(C)C ; [2H][C@@](C)(Nc1c(-c2c(F)cc(F)cc2F)c(Cl)nc2ncnn12)C(C)C]

In other embodiments, the compound is:

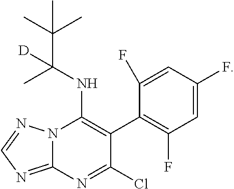 [SMILES: [2H]C(C)(Nc1c(-c2c(F)cc(F)cc2F)c(Cl)nc2ncnn12)C(C)(C)C]

In still further embodiments, the compound is:

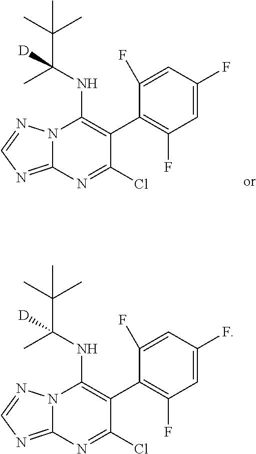 [SMILES: [2H][C@](C)(Nc1c(-c2c(F)cc(F)cc2F)c(Cl)nc2ncnn12)C(C)(C)C ; [2H][C@@](C)(Nc1c(-c2c(F)cc(F)cc2F)c(Cl)nc2ncnn12)C(C)(C)C]

The compounds discussed above may encompass tautomeric forms of the structures provided herein characterized by the bioactivity of the drawn structures. Further, the compounds may also be used in the form of salts derived from pharmaceutically or physiologically acceptable acids, bases, alkali metals and alkaline earth metals.

In some embodiments, pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids.

In other embodiments, pharmaceutically acceptable salts may also be formed from inorganic bases, desirably alkali metal salts including, e.g., sodium, lithium, or potassium, such as alkali metal hydroxides. Examples of inorganic bases include, without limitation, sodium hydroxide, potassium hydroxide, calcium hydroxide, and magnesium hydroxide. Pharmaceutically acceptable salts may also be formed from organic bases, such as ammonium salts, mono-, di-, and trimethylammonium, mono-, di- and triethylammonium, mono-, di- and tripropylammonium, ethyldimethylammonium, benzyldimethylammonium, cyclohexylammonium, benzyl-ammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmono-ethanolammonium, diethanolamine, ethylenediamine, and the like. In one example, the base is selected from among sodium hydroxide, lithium hydroxide, potassium hydroxide, and mixtures thereof.

The disclosure also provides pharmaceutical compositions that contain a compound discussed herein in a pharmaceutically acceptable excipient. In some embodiments, a compound described above is present in a single composition. In other embodiments, a compound described above is combined with one or more excipients and/or other therapeutic agents as described below.

The pharmaceutical compositions include a compound described herein formulated neat or with one or more pharmaceutically acceptable excipients for administration, the proportion of which is determined by the solubility and chemical nature of the compound, chosen route of administration and standard pharmacological practice. The pharmaceutically acceptable excipient may be solid or liquid.

The compound or composition may be administered to a subject by any desirable route, taking into consideration the specific condition for which it has been selected. The compound may, therefore, be delivered orally, by injection, i.e., transdermally, intravenously, subcutaneously, intramuscularly, intravenous, intra-arterial, intraperitoneal, intracavitary, or epiduraly, among others.

Although the compound may be administered alone, it may also be administered in the presence of one or more pharmaceutically acceptable excipient that are physiologically compatible. In some embodiments, the pharmaceutically acceptable excipient is a carrier.

The carrier may be in dry or liquid form and must be pharmaceutically acceptable. Liquid pharmaceutical compositions are typically sterile solutions or suspensions. When liquid carriers are utilized, they are desirably sterile liquids. Liquid carriers are typically utilized in preparing solutions, suspensions, emulsions, syrups and elixirs. In some embodiments, the compound is dissolved a liquid carrier. In some embodiments, the compound is suspended in a liquid carrier. One of skill in the art of formulations would be able to select a suitable liquid carrier, depending on the route of administration. In other embodiments, the liquid carrier includes, without limitation, water, organic solvents, oils, fats, or mixtures thereof. In yet other embodiments, the liquid carrier is water containing cellulose derivatives such as sodium carboxymethyl cellulose. In further embodiments, the liquid carrier is water and/or dimethylsulfoxide. Examples of organic solvents include, without limitation, alcohols such as monohydric alcohols and polyhydric alcohols, e.g., glycols and their derivatives, among others. Examples of oils include, without limitation, fractionated coconut oil, arachis oil, corn oil, peanut oil, and sesame oil and oily esters such as ethyl oleate and isopropyl myristate.

Alternatively, the compound may be formulated in a solid carrier. In some embodiments, the composition may be compacted into a unit dose form, i.e., tablet or caplet. In other embodiments, the composition may be added to unit dose form, i.e., a capsule. In further embodiments, the composition may be formulated for administration as a powder. The solid carrier may perform a variety of functions, i.e., may perform the functions of two or more of the pharmaceutically acceptable excipients described below. For example, the solid carrier may also act as a flavoring agent, lubricant, solubilizer, suspending agent, filler, glidant, compression aid, binder, disintegrant, or encapsulating material. Suitable solid carriers include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. The solid carrier can contain other suitable pharmaceutically acceptable excipients, including those described below.

Examples of pharmaceutically acceptable excipients which may be combined with the compound include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators. See, the excipients described in the “Handbook of Pharmaceutical Excipients”, 5th Edition, Eds.: Rowe, Sheskey, and Owen, APhA Publications (Washington, D.C.), Dec. 14, 2005, which is incorporated herein by reference.

The pharmaceutical composition described herein may be prepared by those skilled in the art. In some embodiments, the pharmaceutical compositions are prepared by combining a compound described herein with a pharmaceutically acceptable excipient.

The compounds described herein are useful in stabilizing microtubules. As such, these compounds are useful in treating diseases that are modulated by microtubules. In some embodiments, the compounds described herein are useful in treating neurodegenerative diseases. Thus, the compounds may be useful in treating neurodegenerative diseases which are characterized by a tauopathy or compromised microtubule function in a subject, such as in the brain of the subject. In some embodiments, the compounds are useful for treating Alzheimer's disease, frontotemporal lobar degeneration, Pick's disease, progressive supranuclear palsy (PSP), corticobasal degeneration, schizophrenia, Parkinson's disease (PD), PD with dementia, Lewy body disease with dementia, amyotrophic lateral sclerosis, argyrophilic grain disease, chronic traumatic encephalopathy, diffuse neurofibrillary tangles with calcification, Down's syndrome, Familial British dementia, Familial Danish dementia, frontotemporal dementia, parkinsonism linked to chromosome 17, Gerstmann-Straussler-Scheinker disease, Guadeloupean parkinsonism, multiple sclerosis, myotonic dystrophy, neurodegeneration with brain iron accumulation, Niemann-Pick disease, type C, non-Guamanian motor neuron disease with neurofibrillary tangles, postencephalitic parkinsonism, prion protein cerebral amyloid angiopathy, progressive subcortical gliosis, SLC9A6-related mental retardation, subacute sclerosing panencephalitis, tangle-only dementia, white matter tauopathy with globular glial inclusions. In other embodiments, the compounds can be used to treat traumatic brain injury (TBI), especially repetitive TBI (rTBI), such as that due to dementia pugilistica and recurrent football concussions and military closed head injuries such as that due to IEDs, which also is known as chronic traumatic encephalopathy (CTE), with features of tauopathy or AD-like pathology or post-traumatic stress disorder. In some embodiments, the compounds can be used to treat chronic traumatic encephalopathy. In other embodiments, the compounds can be used to treat post-traumatic stress disorder. In further embodiments, the compounds are useful in treating Alzheimer's disease. In other embodiments, the compounds are useful in treating schizophrenia.

In other embodiments, the compounds are useful for treating cancer. The term “cancer” as used herein, refers to neoplastic cells in a patient which have abnormal cell group and invade or have the potential to invade one or more body parts of the patient. In some embodiments, the cancer is breast cancer, uterine cancer, lung cancer, ovarian cancer, skin cancer, or non-Hodgkin's lymphoma. In other embodiments, the cancer is breast cancer. In further embodiments, the cancer is uterine cancer. In other embodiments, the cancer is lung cancer. In yet other embodiments, the cancer is ovarian cancer. In still further embodiments, the cancer is skin cancer. In other embodiments, the cancer is non-Hodgkin's lymphoma.

In some embodiments, a therapeutically effective amount of a pharmaceutical agent according to the disclosure is administered to a subject suffering from or diagnosed as having such a disease, disorder, or condition. A “therapeutically effective amount” means an amount or dose sufficient to generally bring about the desired therapeutic benefit in patients in need of such treatment for the designated disease, disorder, or condition. The “therapeutically effective amount” may also mean the amount of the compound to stabilize microtubules. Therapeutically effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

These therapeutically effective amounts may be provided on regular schedule, i.e., daily, weekly, monthly, or yearly basis or on an irregular schedule with varying administration days, weeks, months, etc. Alternatively, the therapeutically effective amount to be administered may vary. In some embodiments, the therapeutically effective amount for the first dose is higher than the therapeutically effective amount for one or more of the subsequent doses. In other embodiments, the therapeutically effective amount for the first dose is lower than the therapeutically effective amount for one or more of the subsequent doses.

Also provided herein are kits or packages containing a compound or composition described herein. The kits may be organized to indicate a single formulation or combination of formulations to be taken at each desired time. The composition may also be sub-divided to contain appropriate quantities of the compound. For example, the unit dosage can be packaged compositions, e.g., packeted powders, vials, ampoules, prefilled syringes or sachets containing liquids.

Suitably, the kit contains packaging or a container with the compound formulated for the desired delivery route. Suitably, the kit contains instructions on dosing and an insert regarding the compound. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the delivery device. Other suitable components to include in such kits will be readily apparent to one of skill in the art, taking into consideration the desired indication and the delivery route. The doses are repeated daily, weekly, or monthly, for a predetermined length of time or as prescribed.

The compound or composition described herein can be a single dose or for continuous or periodic discontinuous administration. For continuous administration, a package or kit can include the compound in each dosage unit (e.g., solution, lotion, tablet, pill, or other unit described above or utilized in drug delivery). When the compound is to be delivered with periodic discontinuation, a package or kit can include placebos during periods when the compound is not delivered. When varying concentrations of a composition, of the components of the composition, or of relative ratios of the compound or other agents within a composition over time is desired, a package or kit may contain a sequence of dosage units, so varying.

A number of packages or kits are known in the art for the use in dispensing pharmaceutical agents for oral use. In some embodiments, the package has indicators for each period. In other embodiments, the package is a labeled blister package, dial dispenser package, or bottle.

The packaging means of a kit may itself be geared for administration, such as an inhalant, syringe, pipette, eye dropper, or other such like apparatus, from which the formulation may be applied to an infected area of the body, such as the lungs, injected into a subject, or even applied to and mixed with the other components of the kit.

The compound or composition of these kits also may be provided in dried or lyophilized forms. When reagents or components are provided as a dried form, reconstitution generally is by the addition of a suitable solvent. It is envisioned that the solvent also may be provided in another packaging means.

The kits may include a means for containing the vials in close confinement for commercial sale such as, e.g., injection or blow-molded plastic containers into which the desired vials are retained.

Irrespective of the number or type of packages, the kits also may include, or be packaged with a separate instrument for assisting with the injection/administration or placement of the ultimate complex composition within the body of an animal. Such an instrument may be an inhalant, syringe, pipette, forceps, measuring spoon, eye dropper or any such medically approved delivery means. Other instrumentation includes devices that permit the reading or monitoring of reactions in vitro.

In some embodiments, pharmaceutical kits are provided and contain a compound of formula (I), (II), (III), (IV), and/or (V). The compound may be in the presence or absence of one or more of the carriers or pharmaceutically effective excipients described above. The kit may optionally contain instructions for administering the compound to a subject having cancer.

### ASPECTS

### Aspect 1. A compound of formula (I):

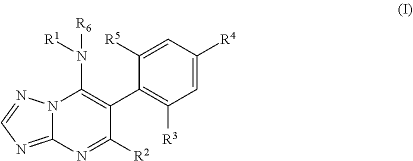

wherein:

R¹ is C₁₋₁₂alkyl, C₃₋₈cycloalkyl, C₁₋₆alk-C₃₋₈cycloalkyl, or C₁₋₆alk-C₃₋₈cycloheteroalkyl, wherein R¹ is substituted by one or more deuterium;

R² is H, Br, Cl, F, CH3, or CF₃;

R³ is H, Br, Cl, or F;

R⁴ is H, Br, Cl, F, CN, CF₃, or CF₂C₁₋₆alkyl; and

R⁶ is H, C₁₋₁₂alkyl, C₃₋₈cycloalkyl, C₁₋₆alk-C₃₋₈cycloalkyl, C₁₋₆alk-C₃₋₈cycloheteroalkyl, diazirinyl, halogenated C₁₋₁₂alkyl, C₁₋₁₂alkyl substituted with diazirinyl, aryl substituted with diazirinyl, or C(O)(aryl substituted with diazirinyl);

R⁵ is H, Br, Cl, or F;

or a pharmaceutically acceptable salt thereof.

Aspect 2. The compound of Aspect 1, wherein R¹ is substituted by one deuterium.

Aspect 3. The compound of Aspect 1 or 2, wherein R¹ is C₁₋₁₂alkyl substituted by one or more deuterium, such as deuterated methyl, deuterated ethyl, deuterated propyl, deuterated butyl, deuterated pentyl, deuterated hexyl, deuterated heptyl, deuterated octyl, deuterated nonyl, or deuterated decyl, preferably deuterated pentyl or deuterated hexyl.

Aspect 4. The compound of any one of the preceding Aspects, wherein R¹ is:

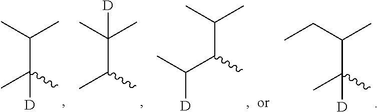

Aspect 5. The compound of any one of the preceding Aspects, wherein R¹ is further substituted by one or more F atoms.

Aspect 6. The compound of any one of the preceding Aspects, wherein R¹ is further substituted by 1 to 3 F atoms.

Aspect 7. The compound of any one of the preceding Aspects, wherein R¹ is further substituted by 1 F atom, such as:

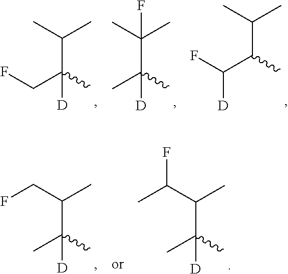

Aspect 8. The compound of any one of Aspects 1 to 6, wherein R¹ is further substituted by 2 F atoms, such as:

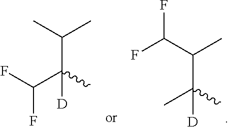

Aspect 9. The compound of any one of Aspects 1 to 6, wherein R¹ is further substituted by 3 F atoms, such as:

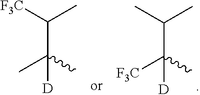

Aspect 10. The compound of Aspect 1 or 2, wherein R¹ is C₃₋₈cycloalkyl substituted by one or more deuterium, such as deuterated cyclopropyl, deuterated cyclobutyl, deuterated cyclopentyl, deuterated cyclohexyl, deuterated cycloheptyl, deuterated cyclooctyl, or deuterated bicyclo[1.1.1]pentyl, preferably deuterated cyclopropyl or deuterated bicyclo[1.1.1]pentyl.

Aspect 11. The compound of Aspect 1 or 2, wherein R¹ is C₁₋₆alk-C₃₋₈cycloalkyl substituted by one or more deuterium, such as deuterated —CH₂-cyclopropyl, deuterated —CH₂-cyclobutyl, deuterated —CH₂-cyclopentyl, deuterated —CH₂-cyclohexyl, deuterated —CH₂-cycloheptyl, deuterated —CH₂-cyclooctyl, or deuterated —CH₂-bicyclo[1.1.1]pentyl, preferably deuterated —CH₂-cyclopropyl or deuterated —CH₂-bicyclo[1.1.1]pentyl.

Aspect 12. The compound of Aspect 11, wherein R¹ is:

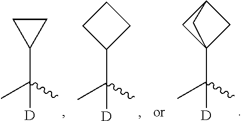

Aspect 13. The compound of any one of Aspects 10 to 12, wherein R¹ is further substituted by 1 to 3 F atoms.

Aspect 14. The compound of Aspect 13, wherein R¹ is further substituted by 1 F atom, such as:

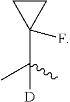

Aspect 15. The compound of Aspect 13, wherein R¹ is further substituted by 3 F atoms, such as:

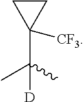

Aspect 16. The compound of Aspect 1 or 2, wherein R¹ is C₁₋₆alk-C₃₋₈cycloheteroalkyl substituted by one or more deuterium, such as deuterated —CH₂-aziridinyl, —CH₂-oxiranyl, —CH₂-thiiranyl, —CH₂-azetidinyl, —CH₂-oxetanyl, —CH₂-thietanyl, —CH₂-pyrrolidinyl, —CH₂-tetrahydrothiophenyl, or —CH₂-tetrahydrofuryl.

Aspect 17. The compound of Aspect 16, wherein R¹ is further substituted by 1 to 3 F atoms, preferably 1 or 3 F atoms.

Aspect 18. The compound of any one of the preceding Aspects, wherein R² is H.

Aspect 19. The compound of any one of Aspects 1 to 17, wherein R² is Br, Cl, or F, preferably Cl.

Aspect 20. The compound of any one of Aspects 1 to 17, wherein R² is CH₃.

Aspect 21. The compound of any one of Aspects 1 to 17, wherein R² is CF₃.

Aspect 22. The compound of any one of the preceding Aspects, wherein R³ is H.

Aspect 23. The compound of any one of Aspects 1 to 21, wherein R³ is Br, Cl, or F, preferably F.

Aspect 24. The compound of any one of the preceding Aspects, wherein R⁴ is H.

Aspect 25. The compound of any one of Aspects 1 to 23, wherein R⁴ is Br, Cl, or F, preferably F.

Aspect 26. The compound of any one of Aspects 1 to 23, wherein R⁴ is CN.

Aspect 27. The compound of any one of Aspects 1 to 23, wherein R⁴ is CF₃.

Aspect 28. The compound of any one of Aspects 1 to 23, wherein R⁴ is CF₂C₁₋₆alkyl.

Aspect 29. The compound of any one of the preceding Aspects, wherein R⁵ is H.

Aspect 30. The compound of any one of Aspects 1 to 28, wherein R⁵ is Br, Cl, or F, preferably F.

Aspect 31. The compound of any one of the preceding Aspects, wherein R⁶ is H.

Aspect 32. The compound of Aspect 1, that is of formula (II):

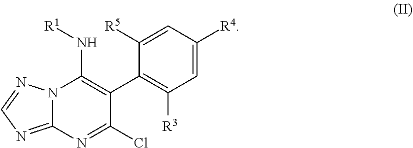

Aspect 33. The compound of Aspect 1, that is of formula (III):

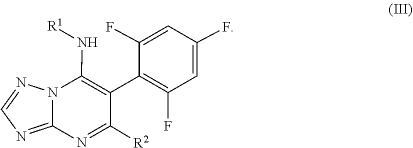

Aspect 34. The compound of Aspect 1, that is of formula (IV):

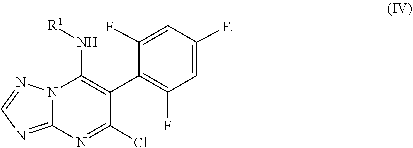

Aspect 35. The compound of Aspect 1, that is of formula (V):

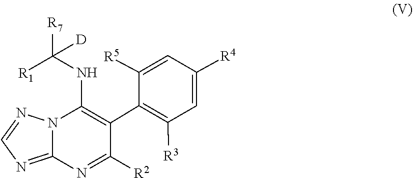

wherein, R⁷ is H, C₁₋₆alkyl, C₃₋₈cycloalkyl, C₁₋₆alk-C₃₋₈cycloalkyl, or C₁₋₆alk-C₃₋₈cycloheteroalkyl.

Aspect 36. The compound of Aspect 1, that is:

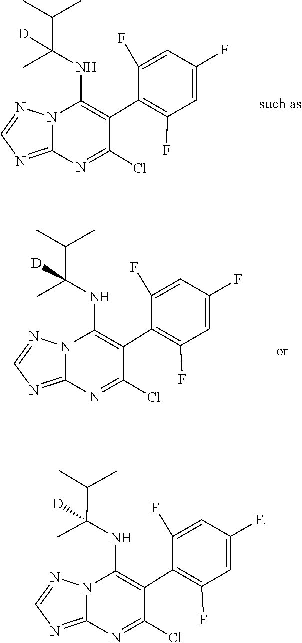 [SMILES: [2H]C(C)(Nc1c(-c2c(F)cc(F)cc2F)c(Cl)nc2ncnn12)C(C)C ; [2H][C@](C)(Nc1c(-c2c(F)cc(F)cc2F)c(Cl)nc2ncnn12)C(C)C ; [2H][C@@](C)(Nc1c(-c2c(F)cc(F)cc2F)c(Cl)nc2ncnn12)C(C)C]

Aspect 37. The compound of Aspect 1, that is:

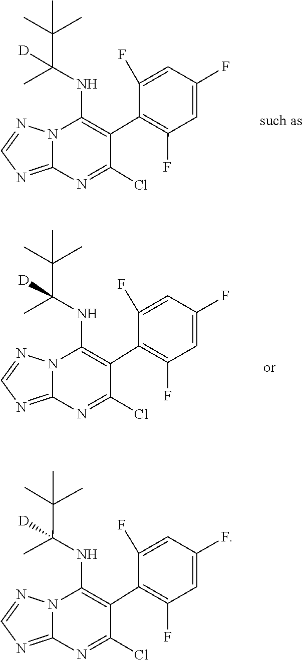 [SMILES: [2H]C(C)(Nc1c(-c2c(F)cc(F)cc2F)c(Cl)nc2ncnn12)C(C)(C)C ; [2H][C@](C)(Nc1c(-c2c(F)cc(F)cc2F)c(Cl)nc2ncnn12)C(C)(C)C ; [2H][C@@](C)(Nc1c(-c2c(F)cc(F)cc2F)c(Cl)nc2ncnn12)C(C)(C)C]

Aspect 38. A composition comprising a compound of any one of the preceding Aspects and a pharmaceutically acceptable excipient.

Aspect 39. A method of stabilizing microtubules in a patient comprising administering to the patient a microtubule-stabilizing amount of a compound of any one of Aspects 1 to 37.

Aspect 40. The method of Aspect 34, wherein the patient has a disease that is a neurodegenerative disease or cancer.

Aspect 41. A method of treating a neurodegenerative disease in a patient comprising administering to the patient a therapeutically effective amount of a compound of any one of Aspects 1 to 37.

Aspect 42. The method of Aspect 41, wherein the neurodegenerative disease is characterized by a tauopathy or compromised microtubule function in the brain of the patient.

Aspect 43. The method of Aspect 41 or 42, wherein the neurodegenerative disease is Alzheimer's disease, frontotemporal lobar degeneration, Pick's disease, progressive supranuclear palsy (PSP), corticobasal degeneration, Parkinson's disease (PD), PD with dementia, Lewy body disease with dementia, or amyotrophic lateral sclerosis.

Aspect 44. The method of Aspect 41 or 42, wherein the neurodegenerative disease is traumatic brain injury, in particular, repetitive traumatic brain injury and chronic traumatic encephalopathy, or post-traumatic stress disorder.

Aspect 45. The method of Aspect 41 or 42, wherein the neurodegenerative disease is schizophrenia.

Aspect 46. A method of treating a cancer in a patient comprising administering to the patient a therapeutically effective amount of a compound of any one of Aspects 1 to 37.

Aspect 47. The method of Aspect 46, wherein the cancer is breast cancer, uterine cancer, lung cancer, ovarian cancer, and skin cancer, or non-Hodgkin's lymphoma.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### EXAMPLES

### Example 1: N-benzyl-3-methylbutan-2-d-2-amine

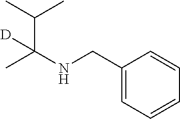

To a solution of 3-methylbutan-2-one (500 mg, 5.81 mmol, 1.00 equiv.) and benzylamine (1.90 mL, 17.40 mmol, 3.00 equiv.) in anhydrous MeOH (14.0 mL) was added Ti(iPrO)₄ (2.23 mL, 7.55 mmol, 1.30 equiv.). The reaction was stirred at room temperature overnight. Then the reaction was cooled to 0° C. and NaBD₄ (243 mg, 5.81 mmol, 1.00 equiv.) was portionwise added. The reaction was stirred 15 minutes at 0° C. and then for 3 hours at room temperature. Water (0.30 mL) was added and stirred for 20 minutes followed by an addition of HCl 1N (1.25 mL). The reaction was filtered over Celite. The Celite was washed with water and EtOAc. The layers were separated and the aqueous layer was washed with EtOAc (×3). The combined organic layers were washed with brine, dried over Na₂SO₄, filtered and concentrated under reduced pressure. The crude product was purified by silica gel column chromatography (0-30% EtOAc in Hexanes) to give the desired product as colorless liquid in 62% yield (638 mg, 3.58 mmol). ¹HNMR (600 MHz, CDCl₃) δ7.35-7.30 (m, 4H), 7.25-7.22 (m, 1H), 3.83 (d, J=13.1 Hz, 1H), 3.72 (d, J=13.1 Hz, 1H), 1.77-1.69 (m, 1H), 1.38 (bs, 1H), 0.99 (s, 3H), 0.90 (d, J=6.8 Hz, 3H), 0.88 (d, J=6.9 Hz, 3H). ¹³C NMR (151 MHz, CDCl₃) δ141.19, 128.48, 128.24, 126.89, 57.08 (t, J=15.8 Hz), 51.60, 32.18, 19.46, 17.33, 15.91. HRMS (ES+) calculated for C₁₂H₁₉DN [M+H]⁺ 179.1653, found 179.1656

### Example 2: N-benzyl-3,3-dimethylbutan-2-d-2-amine

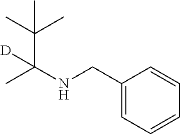

To a solution of 3,3-dimethylbutan-2-one (500 mg, 5.00 mmol, 1.00 equiv.) and benzylamine (1.64 mL, 15.00 mmol, 3.00 equiv.) in anhydrous MeOH (12.0 mL) was added Ti(iPrO)₄ (1.92 mL, 6.50 mmol, 1.30 equiv.). The reaction was stirred at room temperature overnight. Then the reaction was cooled to 0° C. and NaBD₄ (209 mg, 5.00 mmol, 1.00 equiv.) was added portionwise. The reaction was stirred for 15 minutes at 0° C. and then for 3 hours at room temperature. Water (0.30 mL) was added and stirred for 20 minutes followed by an addition of HCl 1N (1.20 mL). The reaction was filtered over Celite. The Celite was washed with water and EtOAc. The layers were separated and the aqueous layer was washed with EtOAc (×3). The combined organic layers were washed with brine, dried over Na₂SO₄, filtered and concentrated under reduced pressure. The crude product was purified by silica gel column chromatography (0-10% EtOAc in Hexanes) to give the desired product as colorless liquid in 51% yield (492 mg, 2.56 mmol). ¹HNMR (600 MHz, CDCl₃) δ7.36-7.30 (m, 4H), 7.25-7.22 (m, 1H), 3.93 (d, J=13.2 Hz, 1H), 3.66 (d, J=13.2 Hz, 1H), 1.01 (s, 3H), 0.88 (s, 9H). ¹³C NMR (151 MHz, CDCl₃) δ141.40, 128.39, 128.30, 126.85, 60.75 (t, J=19.6 Hz), 52.67, 34.46, 26.60, 14.64. HRMS (ES+) calculated for C₁₃H₂₁DN [M+H]⁺ 193.1810, found 193.1813

### Example 3: 3-methylbutan-2-d-2-amine

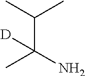

To a solution of N-benzyl-3-methylbutan-2-d-2-amine (130 mg, 0.73 mmol, 1.00 equiv.) in MeOH (22 mL, previously degassed) was added ammonium formate (374 mg) and Pd/C (10% wt.) (205 mg). The reaction was heated at reflux for 1.5 h. The reaction was then cooled to room temperature and filtered over Celite. To the filtrate was added a solution of HCl (2M in Et₂O, 15 mL) and the solution was concentrated to give the desired product as the hydrochloride salt form. ¹H NMR (600 MHz, MeOD) δ1.88 (hept, J=6.6 Hz, 1H), 1.24 (s, 3H), 1.02 (d, J=6.8 Hz, 3H), 0.99 (d, J=6.8 Hz, 3H). ¹³C NMR (151 MHz, MeOD) δ53.71 (t, J=21.1 Hz), 32.65, 18.98, 17.55, 15.26. HRMS (ES+) calculated for C₅H₁₃DN [M+H]⁺ 89.1184, found 89.1185

### Example 4: 3,3-dimethylbutan-2-d-2-amine

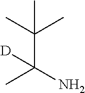

To a solution of N-benzyl-3,3-dimethylbutan-2-d-2-amine (120 mg, 0.62 mmol, 1.00 equiv.) in MeOH (19 mL, previously degassed) was added ammonium formate (320 mg) and Pd/C (10% wt.) (176 mg). The reaction was heated at reflux for 1.5 h. The reaction was then cooled to room temperature and filtered over Celite. To the filtrate was added a solution of HCl (2M in Et₂O, 15 mL) and the solution was concentrated to give the desired product as the hydrochloride salt form. ¹H NMR (600 MHz, MeOD) δ1.26 (s, 3H), 1.02 (s, 9H). ¹³C NMR (151 MHz, MeOD) δ57.30 (t, J=22.6 Hz), 33.87, 26.04, 14.57. HRMS (ES+) calculated for C₆H₁₅DN [M+H]⁺ 103.1340, found 103.1340

### Example 5: 5-chloro-N-(3-methylbutan-2-yl-2-d)-6-(2,4,6-trifluorophenyl)-[1,2,4]triazolo[1,5-a]pyrimidin-7-amine

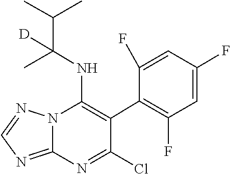 [SMILES: [2H]C(C)(Nc1c(-c2c(F)cc(F)cc2F)c(Cl)nc2ncnn12)C(C)C]

To a solution of 5,7-dichloro-6-(2,4,6-trifluorophenyl)-[1,2,4]triazolo[1,5-a]pyrimidine (50 mg, 0.16 mmol, 1.00 equiv.) and 3-methylbutan-2-d-2-amine hydrochloride (22 mg, 0.17 mmol, 1.10 equiv) in anhydrous DMF (1.70 mL) was added DIPEA (82 μL, 0.47 mmol, 3.00 equiv.). The reaction was stirred at room temperature for 30 minutes. Water was added and extracted with EtOAc (×3). The combined organic layers were washed with water (×2), brine, dried over Na₂SO₄, filtered and concentrated under reduced pressure. The crude product was purified by silica gel column chromatography (0-30% EtOAc in Hexanes) to give the desired racemic product as white solid in 22% yield (13 mg, 0.04 mmol). ¹H NMR (600 MHz, CDCl₃) δ8.30 (s, 1H), 6.84 (t, J=7.4 Hz, 2H), 6.36 (bs, 1H), 1.67-1.57 (m, 1H), 1.04 (s, 3H), 0.78 (t, J=6.8 Hz, 6H). ¹³C NMR (151 MHz, CDCl₃) δ164.02 (dt, J=253.9, 15.1 Hz), 161.50 (ddd, J=250.6, 14.9, 8.4 Hz), 161.38 (ddd, J=250.6, 14.9, 8.4 Hz), 158.06, 154.90, 153.65, 146.05, 107.38 (td, J=20.7, 4.7 Hz), 101.04 (td, J=25.9, 4.1 Hz), 100.99 (td, J=25.9, 4.0 Hz), 88.74, 54.53 (t, J=21.1 Hz), 33.50, 18.09, 17.94, 17.86.

The racemic product was resolved by chiral HPLC to give each enantiomer:

5-chloro-N-(3-methylbutan-2-yl-2-d)-6-(2,4,6-trifluorophenyl)-[1,2,4]triazolo[1,5-a]pyrimidin-7-amine—fast eluting enantiomer: HPLC retention time: 23.01 min; HRMS (ES+) calculated for C₁₆H₁₅DClF₃N₅ [M+H]⁺ 371.1104, found 371.1102.

5-chloro-N-(3-methylbutan-2-yl-2-d)-6-(2,4,6-trifluorophenyl)-[1,2,4]triazolo[1,5-a]pyrimidin-7-amine—slow eluting enantiomer; HPLC retention time: 25.25 min; HRMS (ES+) calculated for C₁₆H₁₅DClF₃N₅ [M+H]⁺ 371.1104, found 371.1103

### Example 6: 5-chloro-N-(3,3-dimethylbutan-2-yl-2-d)-6-(2,4,6-trifluorophenyl)-[1,2,4]triazolo[1,5-a]pyrimidin-7-amine

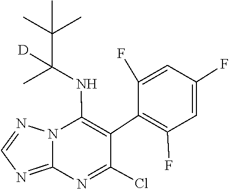 [SMILES: [2H]C(C)(Nc1c(-c2c(F)cc(F)cc2F)c(Cl)nc2ncnn12)C(C)(C)C]

To a solution of 5,7-dichloro-6-(2,4,6-trifluorophenyl)-[1,2,4]triazolo[1,5-a]pyrimidine (60 mg, 0.19 mmol, 1.00 equiv.) and 3,3-dimethylbutan-2-d-2-amine hydrochloride (26 mg, 0.17 mmol, 1.10 equiv) in anhydrous DMF (2.00 mL) was added DIPEA (98 μL, 0.56 mmol, 3.00 equiv.). The reaction was stirred at room temperature for 30 minutes. Water was added and extracted with EtOAc (×3). The combined organic layers were washed with water (×2), brine, dried over Na₂SO₄, filtered and concentrated. The crude product was purified by silica gel column chromatography (0-30% EtOAc in Hexanes) to give the desired racemic product as white solid in 21% yield (15 mg, 0.04 mmol). ¹H NMR (600 MHz, CDCl₃) δ8.33 (s, 1H), 6.87 (t, J=7.6 Hz, 2H), 6.44 (bs, 1H), 1.01 (s, 3H), 0.84 (s, 9H). ¹³C NMR (151 MHz, CDCl₃) δ164.11 (dt, J=254.0, 15.1 Hz), 161.57 (ddd, J=250.9, 14.8, 8.4 Hz), 161.33 (ddd, J=250.7, 14.9, 8.3 Hz), 158.18, 155.01, 153.66, 146.29, 107.59 (td, J=20.5, 4.8 Hz), 101.38-100.78 (m), 88.64, 57.73 (t, J=21.1 Hz), 34.67, 25.82, 16.52.

The racemic product was resolved by chiral chromatography to give each enantiomer:

(R)-5-chloro-N-(3,3-dimethylbutan-2-yl-2-d)-6-(2,4,6-trifluorophenyl)-[1,2,4]triazolo[1,5-a]pyrimidin-7-amine: HPLC retention time: 13.66 minutes; HRMS (ES+) calculated for C₁₇H₁₇DClF₃N₅ [M+H]⁺ 385.1260, found 385.1261

(S)-5-chloro-N-(3,3-dimethylbutan-2-yl-2-d)-6-(2,4,6-trifluorophenyl)-[1,2,4]triazolo[1,5-a]pyrimidin-7-amine: HPLC retention time: 11.75 minutes; HRMS (ES+) calculated for C₁₇H₁₇DClF₃N₅ [M+H]⁺ 385.1260, found 385.1261

### Example 7: Biological Methods

### Microtubule Stabilization Assay

The HEK293 cell subclone, QBI293, was maintained in DMEM supplemented with 10% fetal calf or bovine serum, 1% penicillin/streptomycin antibiotic solution and 1% glutamine at 37° C. in 5% CO₂. Cells were plated into 6-well plates at a density of 800,000 cells/well. Each plate had a well that contained a known microtubule stabilizer as a positive control and the remaining wells were used for compound evaluation. Test compounds were added at multiple concentrations to the test wells and incubated for 4 h. Subsequently, the media was removed and the cells were washed with 1 mL of ice-cold phosphate-buffered saline (PBS). After removal of PBS, 0.2 mL of ice-cold RIPA (0.5% sodium deoxycholate, 0.1% SDS, 1% NP-40, 5 mM EDTA, pH 8.0) containing protease inhibitor mix (1 μg/mL each of pepstatin, leupeptin, TLCK, TPCK and trypsin inhibitor), 1 mM PMSF and 1 μM trichostatin A was added to the wells. The wells were then scraped using a cell scraper and pipetted into 1.5 mL microfuge tubes. The tubes were sonicated with a handheld sonicator at 20× on a power setting of 2, followed by centrifugation for 30 min at 15,000 rpm at 40° C. Supernatants were removed and quantified for protein concentration and the acetyl-tubulin levels were determined using an ELISA as described.

### Acetyl- and Alpha-Tubulin ELISA

The enzyme-linked immunosorbent assay (ELISA) was performed as previously described in Brunden, 2011, Pharmacological Research, 63:341, which is incorporated by reference herein. Briefly, 384-well plates were coated with 12G10 α-tubulin antibody (10 μg/mL; Covance, Princeton, N.J., USA) in 30 μL of cold 0.1 M bicarbonate buffer. 12G10 anti-α-tubulin antibody was originally deposited to the Developmental Studies Hybridoma Bank by Frankel and Nelsen. After overnight incubation at 4° C., the plates were blocked in Block Ace solution (Bio-Rad, Hercules, Calif., USA) for a minimum of 24 hours at 4° C. QBI-293 cell homogenates were diluted in C buffer (0.02 M sodium phosphate, 2 mM EDTA, 0.4 M NaCl, 1% BSA, 0.005% Thimerosal, pH 7.0). Typically, two-fold dilutions from 266 ng/μL to 22.2 ng/μL for QBI cells were performed and 30 μL of sample were added to wells in duplicate. Plates were sealed, centrifuged, and incubated overnight at 4° C. Following incubation with antigen, wells were aspirated and washed with PBS containing 0.05% Tween-20 and 0.005% thimerosal (PBS-Tween buffer). A horseradish peroxidase (HRP)-acetyl-tubulin reporter antibody was prepared by conjugating acetyl-tubulin primary antibody (Sigma Aldrich; clone 6-11B-1) to HRP using a commercially-available peroxidase labeling kit (Roche Applied Science, Indianapolis, Ind., USA). The HRP-acetyl-tubulin reporter antibody (1:1,000 v/v; Sigma Aldrich) or pre-conjugated HRP-alpha-tubulin (1:5,000 v/v; ProteinTech Group, Chicago, Ill., USA) diluted in C buffer were added to appropriate wells (30 μL per well). The plates were sealed and incubated at room temperature for 4 hours on a platform rocker, followed by washing with PBS-Tween buffer. Peroxidase substrate solution (30 μL; KPL, Gaithersburg, Md., USA) was added to each well and the reaction was quenched after 10 min with 10% phosphoric acid (30 μL). Plates were read on a SpectraMax M5 plate reader at an absorbance of 450 nm. The amount of acetyl- and alpha-tubulin protein in each sample was extrapolated using standard curves generated from serial dilutions of known protein standard (acetyl- or alpha-tubulin) concentrations.

### Plasma and Brain Compound Levels

Three young female wild-type mice were injected with test compounds in cassette dosing at 2.5 mg/kg, and sacrificed 1 h later with plasma collection and harvesting of brains. Mouse brains were homogenized in 10 mM ammonium acetate, pH 5.7 (1:2; w/v) using a handheld sonic homogenizer. Mouse plasma was obtained from blood that was collected into a 1.5 mL tube containing 0.5M EDTA solution and which was centrifuged for 10 minutes at 4500 g at 40° C. Aliquots (50 μL) of brain homogenates (50% w/v in 100 mM NH₄OAc pH 5.75) or plasma were mixed with 0.2 ml of acetonitrile, centrifuged at 15,000 g, and the resulting supernatant was used for subsequent LC-MS/MS analysis. Compounds were detected using multiple reaction monitoring (MRM) of their specific collision-induced ion transitions. Samples were separated on an Agilent Zorbax C8 column (3.5 μm, 2.1×50 mm) at 35° C., with mobile phase A of 0.1% (v/v) formic acid, and B of acetonitrile with 0.1% (v/v) formic acid and a gradient of increasing B at 0.4 mL/min. To account for possible matrix effects on analytes, standard curves were generated for each compound from brain homogenate and plasma samples. The standard curve samples were extracted and analyzed in an identical fashion as the corresponding tissue-derived samples, and peak areas were plotted against concentration and a linear regression curve was used to obtain estimated concentrations of the tissue-derived samples using the peak areas.

### Biological Results

The compounds were evaluated in a QBI293 cell-based MT assay as described in Kovalevich et al., J. Pharmacol. Exp. Ther., 2016, 357, 432-50, which is incorporated by reference herein. In summary, acetylated α-tubulin and total α-tubulin levels were determined by ELISA in cell lysates after 4 h of incubation with test compound at 1 and 10 μM (Table 1). In addition, the two most active deuterated compounds were assessed for their ability to partition into the brain after administration to wild-type mice. As shown in Table 1, both of the deuterated compounds showed appreciable brain levels relative to plasma concentration at 1 h after intraperitioneal administration, and both showed greater plasma and brain concentrations than the equivalent non-deuterated analogue when adjusted for dose, suggesting greater metabolic stability of the deuterated compounds.

TABLE 1
Evaluation of deuterated triazolopyrimidines in QBI293 cells to assess for changes in
acetylated-tubulin and α-tubulin relative to vehicle-treated cells

| | | | | Plasma | Brain | |
|---|---|---|---|---|---|---|
| Name | Structure | AcTub | αTub | conc. | conc. | B/P |
| 5-chloro-N-(3- methylbutan-2-yl- 2-d)-6-(2,4,6- trifluorophenyl)- [1,2,4]triazolo[1,5- a]pyrimidin-7- amine (Enantiomer 1) | 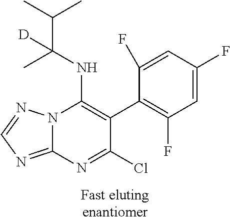 [SMILES: [2H]C(C)(Nc1c(-c2c(F)cc(F)cc2F)c(Cl)nc2ncnn12)C(C)C] | 2.29 ± 0.10 (1 μM) 6.91 ± 0.23 (10 μM) | 1.16 ± 0.04 (1 μM) 1.20 ± 0.01 (10 μM) | 199 ± 46 nM (1 h @ 2.5 mg/kg) 398 nM (scaled to 5 mg/kg) | 551 ± 56 nM (1 h @ 2.5 mg/kg) 1102 nM (scaled to 5 mg/kg) | 2.8 |
| 5-chloro-N-(3- methylbutan-2-yl- 2-d)-6-(2,4,6- trifluorophenyl)- [1,2,4]triazolo[1,5- a]pyrimidin-7- amine (Enantiomer 2) | 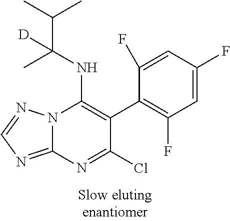 [SMILES: [2H]C(C)(Nc1c(-c2c(F)cc(F)cc2F)c(Cl)nc2ncnn12)C(C)C] | 0.97 ± 0.04 (1 μM) 2.01 ± 0.06 (10 μM) | 0.93 ± 0.05 (1 μM) 0.93 ± 0.02 (10 μM) | | | |
| (R)-5-chloro-N- (3,3- dimethylbutan-2- yl-2-d)-6-(2,4,6- trifluorophenyl)- [1,2,4]triazolo[1,5- a]pyrimidin-7- amine (Enantiomer 1) | 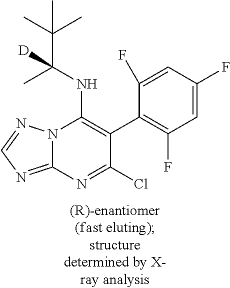 [SMILES: [2H][C@](C)(Nc1c(-c2c(F)cc(F)cc2F)c(Cl)nc2ncnn12)C(C)(C)C] | 5.71 ± 0.13 (1 μM) 7.69 ± 0.49 (10 μM) | 1.00 ± 0.03 (1 μM) 1.05 ± 0.09 (10 μM) | 104 ± 14 nM (1 h @ 2.5 mg/kg) 208 nM (scaled to 5 mg/kg) | 296 ± 48 nM (1 h @ 2.5 mg/kg) 592 nM (scaled to 5 mg/kg) | 2.9 |
| (S)-5-chloro-N- (3,3- dimethylbutan-2- yl-2-d)-6-(2,4,6- trifluorophenyl)- [1,2,4]triazolo[1,5- a]pyrimidin-7- amine (Enantiomer 2) | 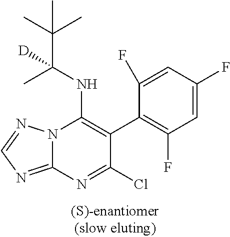 [SMILES: [2H][C@@](C)(Nc1c(-c2c(F)cc(F)cc2F)c(Cl)nc2ncnn12)C(C)(C)C] | 1.10 ± 0.07 (1 μM) 2.92 ± 0.04 (10 μM) | 1.07 ± 0.05 (1 μM) 0.85 ± 0.05 (10 μM) | | | |
| (S)-5-chloro-N- (3,3- dimethylbutan-2- yl)-6-(2,4,6- trifluorophenyl)- [1,2,4]triazolo[1,5- a]pyrimidin-7- amine | 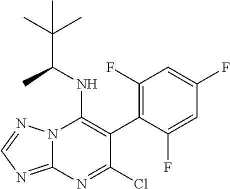 [SMILES: C[C@H](Nc1c(-c2c(F)cc(F)cc2F)c(Cl)nc2ncnn12)C(C)(C)C] | 1.03 ± 0.01 (1 μM) 3.05 ± 0.18 (10 μM) | 0.80 ± 0.06 (1 μM) 0.83 ± 0.06 (10 μM) | | | |
| (R)-5-chloro-N- (3,3- dimethylbutan-2- yl)-6-(2,4,6- trifluorophenyl)- [1,2,4]triazolo[1,5- a]pyrimidin-7- amine | 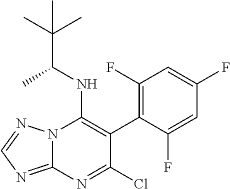 [SMILES: C[C@@H](Nc1c(-c2c(F)cc(F)cc2F)c(Cl)nc2ncnn12)C(C)(C)C] | 5.11 ± 0.47 (1 μM) 7.19 ± 0.31 (10 μM) | 1.00 ± 0.06 (1 μM) 1.03 ± 0.12 (10 μM) | 69 ± 11 nM (1 h @ 5.0 mg/kg) | 153 ± 33 nM (1 h @ 5.0 mg/kg) | 2.2 |
| (S)-5-chloro-N- (3-methylbutan-2- yl)-6-(2,4,6- trifluorophenyl)- [1,2,4]triazolo[1,5- a]pyrimidin-7- amine | 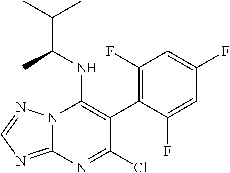 [SMILES: CC(C)[C@H](C)Nc1c(-c2c(F)cc(F)cc2F)c(Cl)nc2ncnn12] | 1.22 ± 0.10 (1 μM) 2.46 ± 0.18 (10 μM) | 0.94 ± 0.04 (1 μM) 0.88 ± 0.02 (10 μM) | | | |
| (R)-5-chloro-N- (3-methylbutan-2- yl)-6-(2,4,6- trifluorophenyl)- [1,2,4]triazolo[1,5- a]pyrimidin-7- amine | 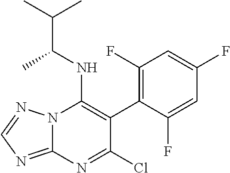 [SMILES: CC(C)[C@@H](C)Nc1c(-c2c(F)cc(F)cc2F)c(Cl)nc2ncnn12] | 2.08 ± 0.09 (1 μM) 2.54 ± 0.10 (10 μM) | 1.10 ± 0.20 (1 μM) 0.91 ± 0.17 (10 μM) | 320 ± 93 nM (1 h @ 5.0 mg/kg) | 812 ± 61 nM (1 h @ 5.0 mg/kg) | 2.5 |

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

1. A compound of formula (I): 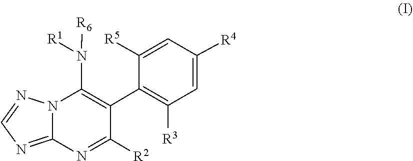
wherein:
R¹ is C₁₋₁₂alkyl, C₃₋₈cycloalkyl, C₁₋₆alk-C₃₋₈cycloalkyl, or C₁₋₆alk-C₃₋₈cycloheteroalkyl, wherein R¹ is substituted by one or more deuterium;
R² is H, Br, Cl, F, CH₃, or CF₃;
R³ is H, Br, Cl, or F;
R⁴ is H, Br, Cl, F, CN, CF₃, or CF₂C₁₋₆alkyl; and
R⁶ is H, C₁₋₁₂alkyl, C₃₋₈cycloalkyl, C₁₋₆alk-C₃₋₈cycloalkyl, C₁₋₆alk-C₃₋₈cycloheteroalkyl, diazirinyl, halogenated C₁₋₁₂alkyl, C₁₋₁₂alkyl substituted with diazirinyl, aryl substituted with diazirinyl, or C(O)(aryl substituted with diazirinyl);
R⁵ is H, Br, Cl, or F;
or a pharmaceutically acceptable salt thereof.

2. The compound of claim 1, wherein R¹ is substituted by one deuterium.

3. The compound of claim 1, wherein R¹ is C₁₋₁₂alkyl substituted by one or more deuterium.

4-5. (canceled)

6. The compound of claim 1, wherein R¹ is further substituted by one or more F atoms.

7-13. (canceled)

14. The compound of claim 1, wherein R¹ is C₃₋₈cycloalkyl substituted by one or more deuterium.

15-16. (canceled)

17. The compound of claim 1, wherein R¹ is C₁₋₆alk-C₃₋₈cycloalkyl substituted by one or more deuterium, optionally further substituted by 1 to 3 F atoms.

18-24. (canceled)

25. The compound of claim 1, wherein R¹ is:

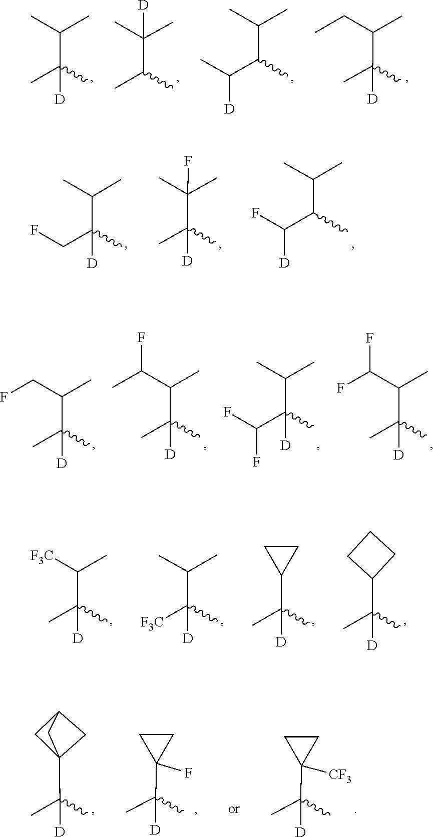

26. The compound of claim 1, wherein R¹ is C₁₋₆alk-C₃₋₈cycloheteroalkyl substituted by one or more deuterium, optionally substituted by 1 to 3 F atoms.

27-28. (canceled)

29. The compound of claim 1, wherein R², R³, R⁴, R⁵, or R⁶ is H.

30. The compound of claim 1, wherein R², R³, R⁴, or R⁵ is Br, Cl, or F.

31. (canceled)

32. The compound of claim 1, wherein R² is CH₃.

33. The compound of claim 1, wherein R² or R⁴ is CF₃.

34-39. (canceled)

40. The compound of claim 1, wherein R⁴ is CN.

41. (canceled)

42. The compound of claim 1, wherein R⁴ is CF₂C₁₋₆alkyl.

43-46. (canceled)

47. The compound of claim 1, that is of formula (II), (III), (IV), or (V): 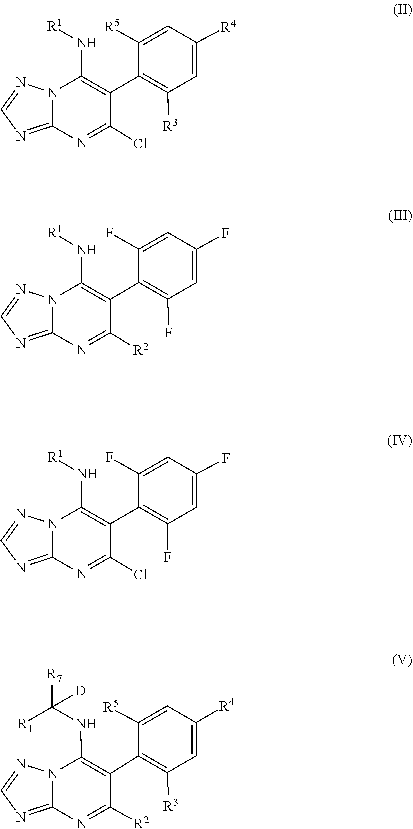
wherein, R⁷ is H, C₁₋₆alkyl, C₃₋₈cycloalkyl, C₁₋₆alk-C₃₋₈cycloalkyl, or C₁₋₆alk-C₃₋₈cycloheteroalkyl.

48-50. (canceled)

51. The compound of claim 1, that is:

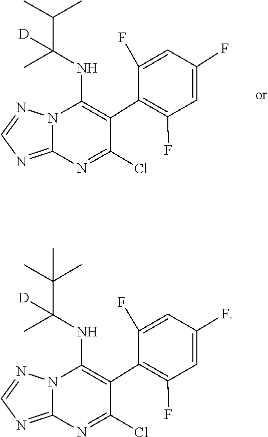 [SMILES: [2H]C(C)(Nc1c(-c2c(F)cc(F)cc2F)c(Cl)nc2ncnn12)C(C)C ; [2H]C(C)(Nc1c(-c2c(F)cc(F)cc2F)c(Cl)nc2ncnn12)C(C)(C)C]

52. The compound of claim 52, that is:

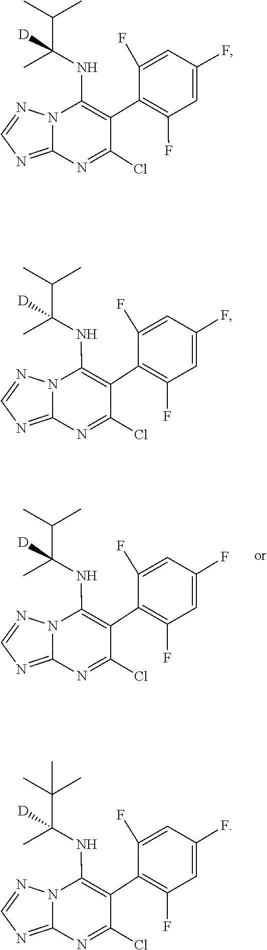 [SMILES: [2H][C@](C)(Nc1c(-c2c(F)cc(F)cc2F)c(Cl)nc2ncnn12)C(C)C ; [2H][C@@](C)(Nc1c(-c2c(F)cc(F)cc2F)c(Cl)nc2ncnn12)C(C)C ; [2H][C@](C)(Nc1c(-c2c(F)cc(F)cc2F)c(Cl)nc2ncnn12)C(C)C ; "or" ; [2H][C@@](C)(Nc1c(-c2c(F)cc(F)cc2F)c(Cl)nc2ncnn12)C(C)(C)C]

53-54. (canceled)

55. A composition comprising a compound of claim 1 and a pharmaceutically acceptable excipient.

56. A method of stabilizing microtubules in a patient comprising administering to the patient a microtubule-stabilizing amount of a compound of claim 1.

57. (canceled)

58. A method of treating a neurodegenerative disease or cancer in a patient comprising administering to the patient a therapeutically effective amount of a compound of claim 1.

59. The method of claim 59, wherein the neurodegenerative disease is:
(i) characterized by a tauopathy or compromised microtubule function in the brain of the patient;
(ii) Alzheimer's disease, frontotemporal lobar degeneration, Pick's disease, progressive supranuclear palsy (PSP), corticobasal degeneration, Parkinson's disease (PD), PD with dementia, Lewy body disease with dementia, or amyotrophic lateral sclerosis;
(iii) traumatic brain injury such as repetitive traumatic brain injury and chronic traumatic encephalopathy, or post-traumatic stress disorder; or
(iv) schizophrenia.

60-63. (canceled)

64. The method of claim 59, wherein the cancer is breast cancer, uterine cancer, lung cancer, ovarian cancer, skin cancer, or non-Hodgkin's lymphoma.
